(19) United States
(12) Patent Application Publication
(10) Pub. No.: US 2007/0219449 A1
(43) Pub. Date: Sep. 20, 2007

(54) Apparatus for Examining a Body of Living Tissues

(76) Inventor: David J. Watmough, Inverness (GB)

Correspondence Address: John L. Cordani;Carmody & Torrance, 50 Leavenworth Street, P.O.Box 1110, Waterbury, CT, 06721-1110, US

(21) Appl. No.: 11/579,846

(22) PCT Filed: May 10, 2005
(86) PCT No.: PCT/GB05/01777

§ 371 Date: Nov. 8, 2006

(30) Foreign Application Priority Data
May 10, 2004 (GB) 0410320.6
Feb. 8, 2005 (GB) 0502524.2

(51) Int. Cl. A61B 8/06

(52) U.S. Cl. 600/454

(57) ABSTRACT

A handheld battery powered apparatus for examining a body of living tissues, which tissues may contain a neoplasm, utilizing a light source for illuminating the tissues comprising a D shaped array of high intensity light emitting diodes overlaid by an optically transparent window, means for reproducing the intensity of the light, means for applying ultrasound to the neoplasm positioned in the centre of the array, means for receiving reflected ultrasound from blood vessels in the tissues also situated in the D shaped array, and a loudspeaker or other means for reproducing the blood flow signals.

The present invention relates to an apparatus for examining a body of living tissues, in particular the female breast or male testicular tissues.

The principal health concern of women is breast cancer. Why? Because it is the commonest cause of death in women under 50 years of age. In the UK there are 41,000 new cases per year and the trend is upwards. There are 13,000 deaths per year from breast cancer. The 5 year survival is 73%. This means 1 in 4 women die within 5 years of diagnosis. The appropriate management of patients with early disease reduces mortality. Presently 15% of women present with advanced disease. Earlier detection should also reduce the number of mastectomies in favour of breast conserving surgery. What can be done to ensure that women with the disease present at an early stage for diagnosis and treatment?

Similar problems concern testicular cancer in men.

The present applicant has developed an easy to use, light-based product, the breast checker, which when employed together with breast self examination is designed to indicate the need for women to consult a GP for advice and possible referral to a hospital setting. We refer to our co-pending patent applications UK 0105471.7 [published as GB 2375672A ] and International Patent Application No. PCT /GB/02/01159[published as WO 03/077744A].

The applicant's prior breast checker is small, safe, simple to use and is purposely chosen to be non-diagnostic. It involves no ionising radiation, no UV radiation, or breast compression. The applicant has also developed a more sophisticated version of the breast checker for use by GPs which employs an optical/ultrasound Doppler probe. Errors sometimes lead to claims for compensation. The price of such failure can exceed £300,000. The clinical breast checker should minimize false reassurance and thus cut the error rate.

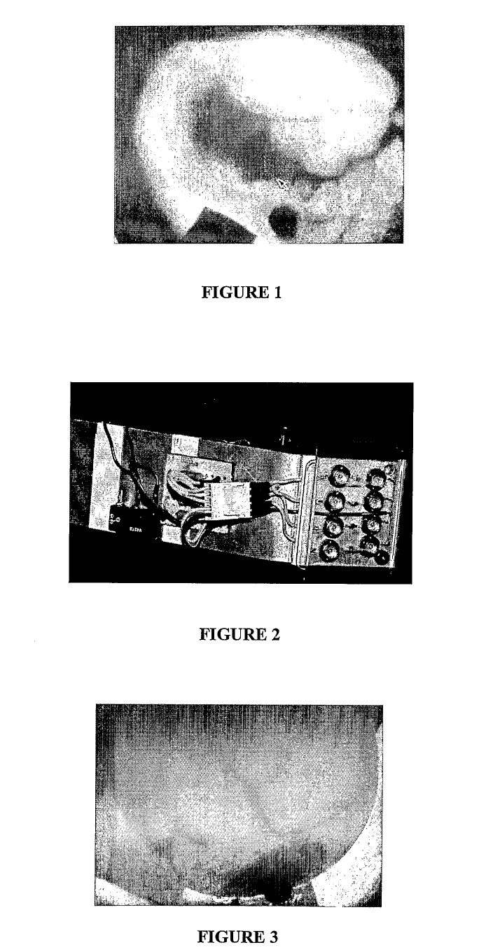

A typical image of an advanced breast cancer is shown by the dark area in the accompanying FIG. 1. The fact that angiogenesis is responsible for the excess light absorption is supported by the presence of the connected dilated vessels seen to the right of the nipple [small dark circle].

FIG. 1 shows an advanced cancer of the breast (arrowed) obtained by means of light transmitted through the tissues from a light guide held against the inferior breast surface.

This present invention aims to provide improvements to the breast checker instruments described in our earlier co-pending applications. In those applications the light source was envisaged as typically a quartz halogen 75 Watt bulb which requires a current of about 6 amperes at 12 volts and thus necessitates a cooling fan since the device can be switched on for extended periods. This increases the minimum size of the device thus requiring separate probe and control box. For prolonged use in a clinical setting mains power is the only practical option. The direct use of mains voltage [240 Volts] in the probe containing the light source is considered undesirable in an instrument in contact with the human body. The instruments described here are all used in a darkened room so that variations in brightness on the surface of a breast through which light is transmitted may be observed.

Ideally a totally self contained hand held unit powered by means of typically 3 or 4 AA type rechargeable batteries is desirable but difficult to implement in practice. The unit requires to provide light of sufficient intensity to transmit a few percent (of that light incident upon it) through an essentially conical tissue volume and to thus provide as nearly as possible, uniform illumination on the exit side [usually the superior aspect of the breast]. Non uniform illumination prevents detection of brightness variations indicative of the presence of small deep malignant tumors. Without use of a tissue compression system, [which is particularly undesirable for incorporation into a domestic device because of both cost and safety] the breast generally approximates to a conical volume, the apex being centred on the nipple.

The problem therefore arises as to how to achieve such uniform brightness over the breast surface in normal breasts (where there is no neoplastic tumor present).

The applicant initially tried a number of light emitting diodes, [LEDs] first in a 4×4 array as shown in FIG. 2. The preferred LEDs are 22 Lumens per Watt high intensity devices.

FIG. 2 shows a first arrangement of 8 LEDs used to demonstrate the possibility of illuminating breast tissues from the inferior aspect with these multiple light sources.

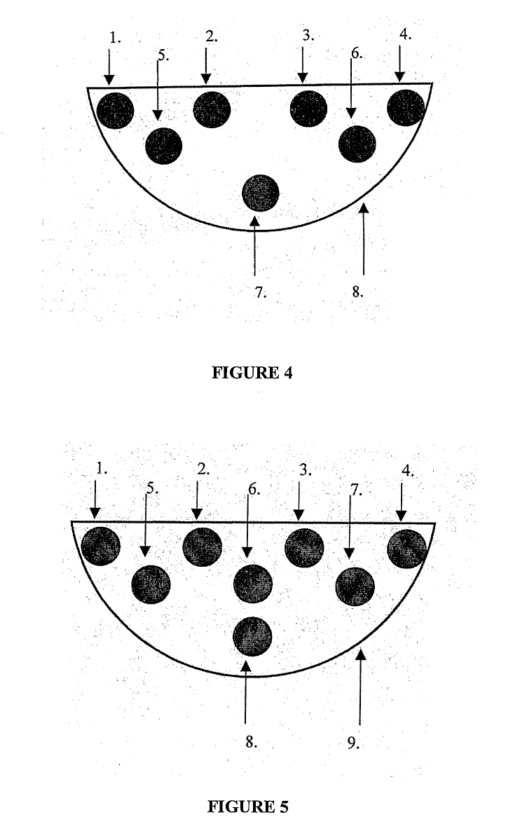

FIG. 3 shows an image of a breast obtained using apparatus shown in FIG. 2 showing serious backscatter of light below and around the periphery of the breast.

With 8 1 Watt LEDs as shown in FIG. 2 there is excessive scatter of lights laterally, medially and below the breast. This will prevent a woman using the breast checker from seeing variations in brightness on the superior aspect of her breast. It is still a problem if she uses a mirror to observe her breasts while applying the breast checker.

The present invention aims to overcome this problem.

Accordingly, the present invention provides a handheld battery powered apparatus for examining a body of living tissues, which tissues may contain a neoplasm, utilizing a light source for illuminating the tissues comprising a D shaped array of high intensity light emitting diodes overlaid by an optically transparent window, means for reproducing the intensity of the light, means for applying ultrasound to the neoplasm positioned in the centre of the array, means for receiving reflected ultrasound from blood vessels in the tissues also situated in the D shaped array, and a loudspeaker or other means for reproducing the blood flow signals.

Preferably, the means for applying ultrasound and means for receiving reflected ultrasonic signals comprises an ultrasonic Doppler blood flow detector whose piezoelectric transmitting and receiving elements are semicircular discs or rectangles positioned in the space provided between the light emitting diodes.

Preferably, the piezoelectric elements are attached to the optically and ultrasonically transparent window overlying the light emitting diodes.

Preferably, the apparatus further comprises a switching device to which the said means for applying light and ultrasound to the tissues being interrogated can be selected. When the optical system comprising the D-shaped array of light emitting diodes reveals an area of darkness on the superior aspect of the breast to the user caused by optical absorption in the tissues, the switch may be moved to permit ultrasonic interrogation of that region of the tissue giving rise to the absorption.

Preferably, the time varying Doppler frequency shift which is electronically produced is revealed to the user via an audio system such as internal or external loudspeaker or headphones.

Preferably, the combined optical ultrasound Doppler device includes an array of 2 or more piezoelectric elements for blood flow detection and analysis, operating at ultrasonic frequencies between 2 and 20 MHz and where the light emitting diodes operate at a wavelength lying between 550 and 700 nm.

Preferably, the intensity of the optical radiation is electronically incremented in steps which are indicated digitally or otherwise to the user so that serial examinations lead to images of tissues comparison of which permits the user to spot changes to the appearance of the illuminated tissue surface caused by underlying pathology at an early stage in the disease process.

Preferably, the light level is held at a low level until a pressure or capacitive sensor incorporated in the device touches the skin whereupon the light level increases to operate at the preset level thus protecting the eyes of the user and allowing them to maintain visual sensitivity. The device may be protected from overheating by a heat sink situated behind the light emitting diodes and an embedded thermostatic element which at a selected temperature interrupts the current driving the diodes.

Preferably, the optical features are included but the ultrasonic features are disabled or omitted.

Preferably, the device has an output socket connectable to an external recording system able to present an ultrasonic Doppler frequency shift spectra.

Preferably, the apparatus is adapted to the examination of testicular tissues.

Preferably, contrast media are injected into the vascular system to increase the sensitivity of the Doppler ultrasound and optical interrogation of tissues.

Preferably, transparent gel or lubricant for enhancing optical penetration of tissues is applied between the window and the skin and through which light and ultrasound are transmitted.

The present invention also provides an apparatus for examining a body of living tissues, the apparatus comprising a light source for illuminating the tissues, the light source having a light emitting face substantially in the shape of an angular segment of a circle.

Preferably, the light emitting face is substantially semi-circular, more preferably substantially D shaped.

Preferably, the light source comprises an array of light emitting diodes.

Preferably, the array of light emitting diodes is substantially D shaped.

Preferably, the light emitting face comprises a window which is transparent to radiation emitted from the light source.

The apparatus may further comprise a control for reproducing the intensity of the light from the light source.

The apparatus may further comprise a means for applying ultrasound to a neoplasm to be examined by the apparatus, a means for receiving reflected ultrasound from blood vessels in the tissues, and a means for generating an output to be analyzed by a user which reproduces blood flow signals derived from the reflected ultrasound signals.

Preferably, the means for applying ultrasound and the means for receiving reflected ultrasound are positioned within the area of the light emitting face, more preferably within a centre of the light emitting face.

Preferably, the means for applying ultrasound and means for receiving reflected ultrasound comprises an ultrasonic Doppler blood flow detector having piezoelectric transmitting and receiving elements positioned in a space provided between the light emitting diodes.

Preferably, the piezoelectric elements are attached to the window overlying the light emitting diodes.

The apparatus may further comprise a switching device to which the said means for applying light and ultrasound to the tissues being interrogated can be selected.

The apparatus may further comprise an audio system by means of which a time varying Doppler frequency shift which is electronically produced is revealed to the user.

Preferably, the means for applying ultrasound and the means for receiving reflected ultrasound includes an array of two or more piezoelectric elements for blood flow detection and analysis, operating at ultrasonic frequencies between 2 and 20 MHz and where the light emitting diodes operate at a wavelength lying between 550 and 700 nm.

The apparatus may further comprise a controller for tie light source whereby the intensity of the optical radiation is electronically incremented in steps which are indicated to the user.

The apparatus may further comprise a light level controller whereby the light level is held at a low level until a pressure or capacitive sensor incorporated in the device touches the skin whereupon the light level increases to operate at the preset level.

The apparatus may further comprise a heat sink situated behind the light emitting diodes and an embedded thermostatic element which at a selected temperature interrupts the current driving the diodes to protect the apparatus from overheating.

The apparatus may further comprise an output socket connectable to an external recording system able to present an ultrasonic Doppler frequency shift spectra.

The apparatus may further comprise a housing for the light source, the light emitting face having a linear edge adjacent to a linear side of the housing and a curved edge projecting away from the linear edge and located inwardly of a side of the housing, whereby the linear edge can be placed substantially against the skin of a user beneath the breast and substantially against the chest wall for illuminating the breast.

Preferably, the linear edge is about 5 mm inwardly of the linear side of the housing.

Preferably, the housing has an integral handle portion remote from the light emitting face.

The present invention also relates to the use of the apparatus according to the present invention for the examination of breast or testicular tissues.

Preferably, contrast media are injected into the vascular system to increase the sensitivity of the Doppler ultrasound and optical interrogation of tissues.

Preferably, transparent gel or lubricant for enhancing optical penetration of tissues is applied between the window and the skin and through which light and ultrasound are transmitted.

Embodiments of the present invention will now be described by ay of example only with reference to the accompanying drawings, in which:

FIG. 1 shows an advanced cancer of the breast (arrowed) obtained by means of light transmitted through the tissues from a known light guide held against the inferior breast surface;

FIG. 2 shows a first arrangement of 8 LEDs, not in accordance with the present invention, used to demonstrate the possibility of illuminating breast tissues from the inferior aspect with these multiple light sources;

FIG. 3 shows an image of a breast obtained using apparatus shown in FIG. 2 showing serious backscatter of light below and around the periphery of the breast;

FIG. 4 shows an array of light emitting diodes in D formation incorporated in an apparatus for examining a body of living tissues in accordance with a first embodiment of the present invention;

FIG. 5 shows an array of light emitting diodes in D formation incorporated in an apparatus for examining a body of living tissues in accordance with a second embodiment of the present invention;

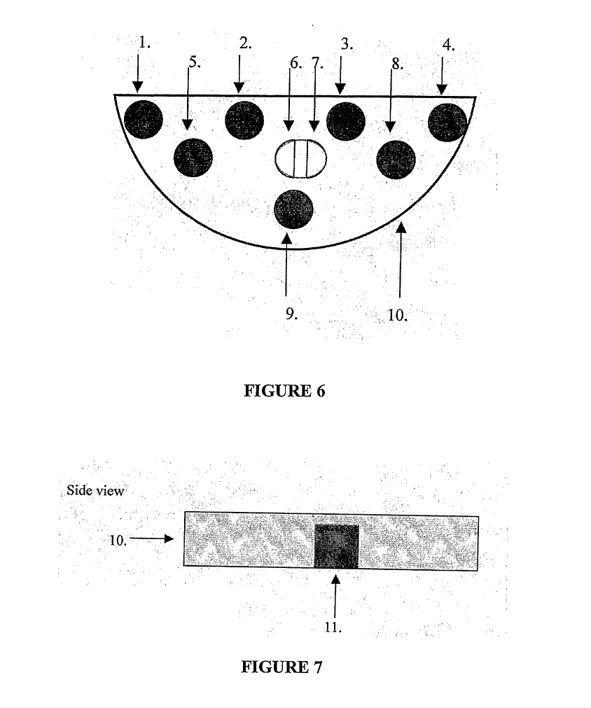

FIG. 6 shows an array of light emitting diodes in D formation incorporated in an apparatus for examining a body of living tissues in accordance with a third embodiment of the present invention;

FIG. 7 shows an optically transparent window which covers the LED array having a circular recess into which the piezoelectric transducers are fixed for incorporation into any of the embodiments of the present invention;

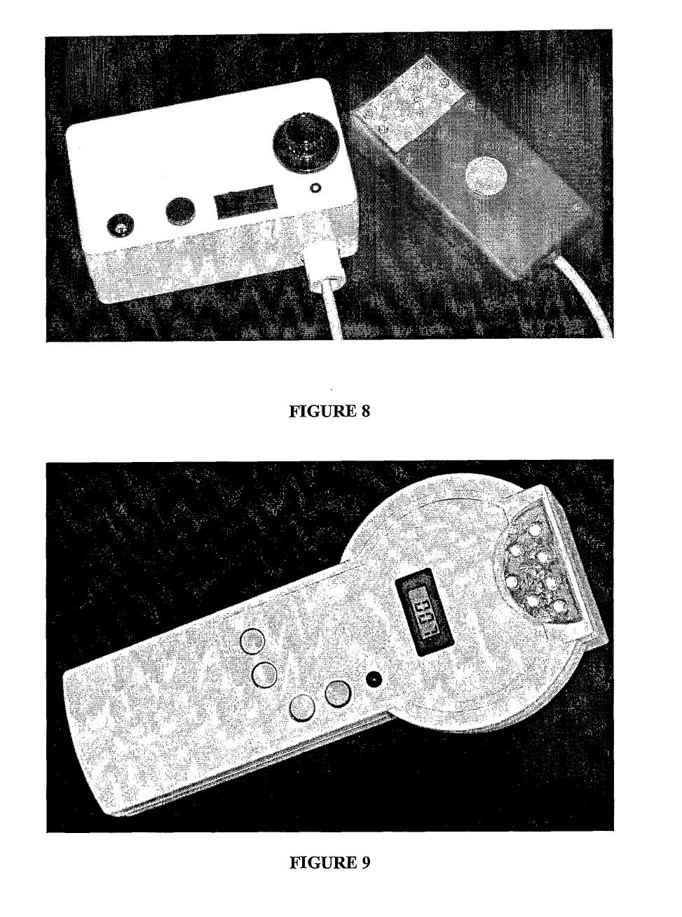

FIG. 8 show an apparatus in accordance with a fourth embodiment of the present invention with a rectangular window including 7 LEDs in D shaped configuration;

FIG. 9 shows a hand held breast checker in accordance with a fifth embodiment of the present invention with 7 LEDs in D-shaped array with D-shaped transparent window;

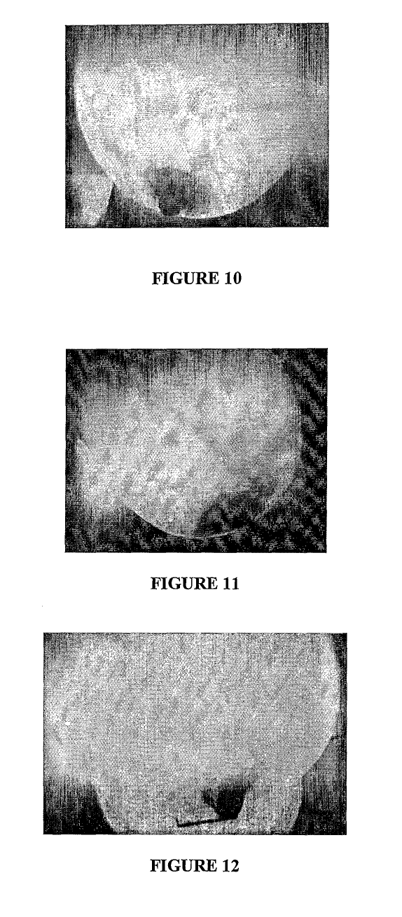

FIGS. 10 and 11 are images of right (R) and left (L) breasts obtained using the apparatus of FIG. 8;

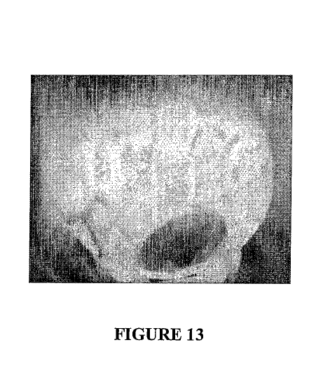

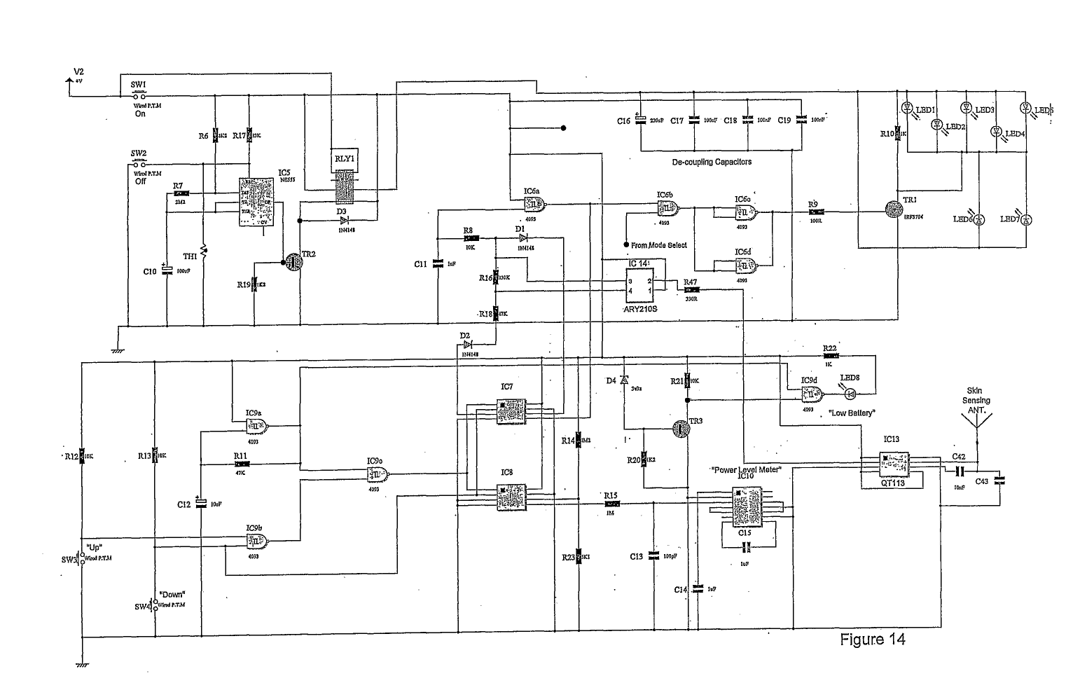

FIGS. 12 and 13 are images of right (R) and left (L) breasts obtained using the apparatus of FIG. 9;

FIG. 14 shows circuitry that controls the LEDs in the device shown in FIG. 9;

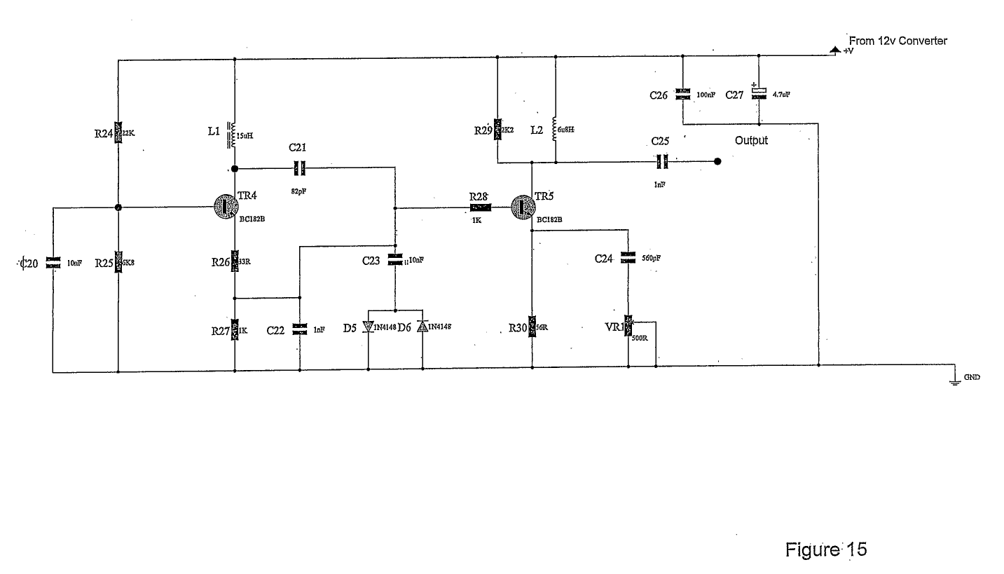

FIG. 15 shows circuitry of the Doppler ultrasound transmitter in the device shown in FIG. 9; and

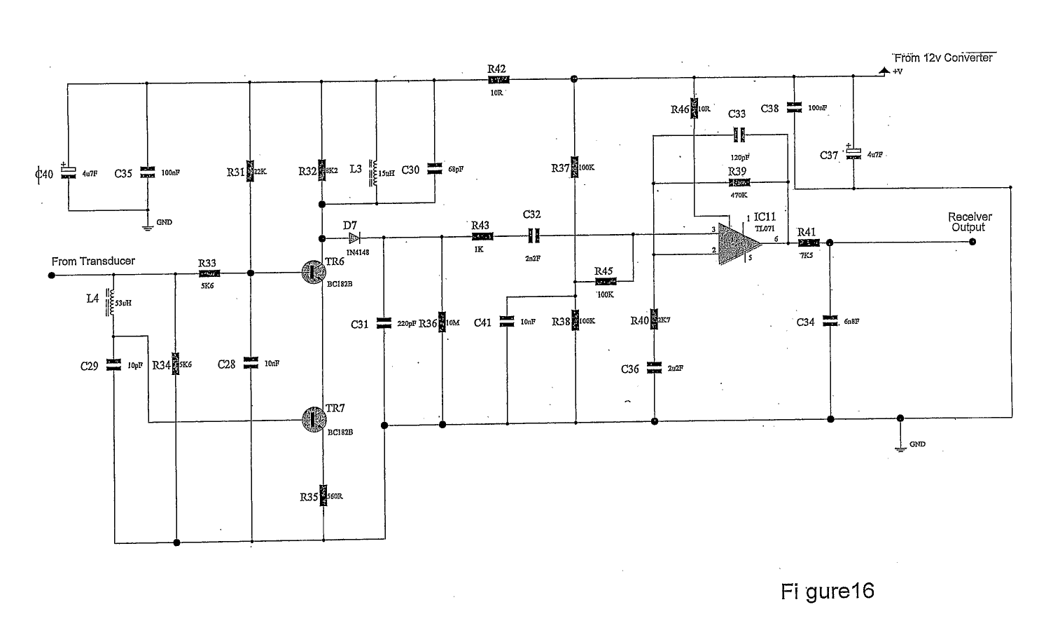

FIG. 16 shows circuitry of the Doppler ultrasound receiver in the device shown in FIG. 9.

Experiments demonstrated that a D-shaped array of LEDs, as shown in the apparatus of FIG. 4, dramatically reduced this problem. To increase the intensity of light transmitted through the breast tissues 8 LEDs can be used as shown in FIG. 5 and still retain the D configuration.

FIG. 4 shows 7 light emitting diodes in D formation. The preferred dimensions are 45 mm chord length and 20 mm in width. LEDs arrowed 1-7. 8 transparent window.

FIG. 5 shows 8 light emitting diodes in a D formation. The preferred dimensions are 45 mm along the chord and 20 mm in width. LEDs arrowed 1-8. Transparent window 9.

FIG. 6 shows a similar arrangement of LEDs to that of FIG. 4 but 2 D shaped piezoelectric elements are diameter around 8 mm labelled 6 and 7, inserted in the window above the LED array. Connecting wires to these transducers are now shown. LEDs arrowed 1,2,3,4,5,8 and 9. Transparent window 10.

FIG. 7 shows an optically transparent window which covers the LED array having a circular recess into which the piezoelectric transducers are fixed. The layer of material above the transducers to which these elements are attached is minimized to around typically 0.2 mm. Window is labelled 10 and the well for housing the piezoelectric transducer elements is labelled 11.

In course of our experiments we also demonstrated the need for a row of LEDs to be placed close to the chest wall to ensure useful examination of the entire breast volume. The separation of 10 mm shown in FIG. 2 is too great and was reduced to about 5 mm in the apparatus shown in FIG. 6.

Another advantage of the D-shaped array of LEDs is the ability to include piezoelectric transducers as shown in FIG. 6 without reducing the light transmitted into tissues. One piezoelectric element transmits ultrasound [pulsed or continuous wave] into breast tissues. Where there is a cancer and associated angiogenesis the sound is scattered by red blood cells and this scattered sound is received by the second piezoelectric element. The frequency shift df of the ultrasonic wave contains information about the blood flow velocities in the angiogenesis vessels and associated arterio venous shunts. Note that df=2 v f cos q/c where v is the flow velocity, f is the transmitted frequency, c is the speed of sound and q is the angle between flow direction and that of the interrogating wave. Our preferred method of including the transducers is to remove a portion of the transparent window (10) which encloses the LEDs. This cavity is numbered 11 in FIG. 7. The preferred thickness of the residual window is 0.2 mm to minimize ultrasonic absorption in the material comprising the window. The transducers may be glued onto the upper face of the cavity which is typically 8 mm in diameter. The rear face of the transducers is preferably air backed for maximum sensitivity though a tungsten loaded epoxy based material may be used as backing material. The preferred frequency of the ultrasound is between 2 and 15 MHz. Lower frequencies ensure sufficient penetration of tissues to reach typical tumors.

A further advantage of the invention described here is that the transducers and associated electronics can be operated while light is passing through tissues. This means that the ultrasonic waves can be directed at the tumor, guided by its optical image, to determine whether or not a tumor is vascularised. The mains operated versions prove more difficult in this respect because electrical noise generated by the fan [referred to above] ‘over power’ the Doppler ultrasound signals. The Doppler signals in the audio frequency range may be extracted electronically, amplified and presented via a mini-loudspeaker in the unit. Characteristic pulsatile sounds from a tumor can be heard by the user when present. Such signals are another indicator of the need to seek medical advice. Note that coupling gel such as KY gel placed on the window does not inhibit imaging but enhances the ultrasonic aspect of the examination.

FIG. 8 show the apparatus with a rectangular window including 7 LEDs in D shaped configuration. The (white) unit connected to the array contains pulse width modulation electronic circuitry and a calibrated control [black knob with scale] to allow repeatability of the light intensity used in one examination to be reproduced in the next. The system is operated with rechargeable batteries.

FIG. 9 shows a hand held breast checker with 7 LEDs in D-shaped array with D-shaped transparent window. There are facilities to increment the light intensity in typically 20 steps up and 20 steps down from the 50% starting level. There is a digital display to show the light level. A memory chip can reset the unit to give the same intensity at next use. A low battery warning light flashes at a predetermined voltage, typically after 30 minutes use. Undue internal heating from prolonged use of the device is obviated by a thermal cut out set at typically 60 degrees Centigrade. Note the need for a very narrow separation between the position of the 4 LEDs [in a line] and the terminal end of the instrument which is normally placed next to the chest wall when in use.

In FIG. 9 a light only unit is shown with 7 LEDs configured in a D-shape. There are on and off buttons plus buttons to increase and decrease the light intensity for optimum light intensity which is not normally maximum intensity. Failure to appreciate this latter point can cause scattering around a tumor to a degree which suppresses the tumor generated optical absorption thus foiling the objective of using the device. The D shaped window and LEDs in FIG. 7 is arranged to be as close as possible to the chest wall of a user. In the preferred device, not shown, the window is curved to fit the curvature of a typical chest wall and for use by doctors or other clinical personnel the Doppler system is included within the hand held device. The unit shown in FIG. 9 contains 3 AA batteries, runs for over 30 minutes and is supplied with a separate charger unit.

In the preferred unit the red LEDs operate at about 630 nanometres. Red/orange and Amber LEDs [617 and 590 nm respectively] may be used but the tissues are less transparent at those wavelengths and higher light intensities maybe required to produce useful breast images.

There is a low battery warning light indicating the need for connection to recharging circuitry. An LCD display or stack of low power LEDs shows the light intensity on a scale of 0-100%. A facility to disable the breast checker while being recharged from the mains is desirable and is included in the preferred embodiment.

FIGS. 10 and 11 are images of R and L breasts obtained using the apparatus of FIG. 8. Superficial blood vessels, areola and nipples are demonstrated and only a modest light component is scattered laterally and there is virtually non in the forward direction.

FIG. 12 shows how increasing the light transmission through tissues beyond optimum decreases contrast so that the blood vessels become barely visible. FIG. 13 shows an image produced by the apparatus described in FIG. 9 and demonstrates excellent contrast between blood vessels and tissue, however there is forward light scatter because of the depression in the casing next to the D-shaped window. This depression is omitted in the preferred instrument outlined in this patent application.

The clarity of superficial vessels is a marker for obtaining good images of angiogenesis at a depth in tissue.

FIG. 10 shows an image of R breast obtained with apparatus of FIG. 8.

FIG. 11 shows an image of L breast obtained with apparatus of FIG. 8.

FIG. 12. Image to show over exposed image of R breast with light of intensity set at a level which is too high. Neoplastic tumors will not show up in these circumstances. The apparatus shown in FIG. 9 was used.

FIG. 13. Image of L breast obtained with apparatus of FIG. 9 set at the correct intensity level.

The sensitivity of more primitive earlier versions of the apparatus described here demonstrated sensitivity for detecting cancer of 0.94 and 0.73 Bundred et al (1986) and Brittenden et al (1995). One of the present inventors first showed that light passing through normal and pathological breast tissue samples demonstrated cancer because of associated angiogenesis, Watmough (1982). Thus when a cancer is demonstrated by the breast checker, there is angiogenesis. It follows that the cancer is in its exponentially growing phase and is therefore life threatening. Some microscopic cancers without angiogenesis may be dormant and never surface clinically in the patient's life time. This fact emphasizes the value of the breast checker since microscopic cancers without angiogenesis will fail to be revealed. This is a strength rather than a weakness.

The preferred LEDs used to obtain the images presented here operate at 627 nanometres but similar devices which operate at 617 and 590 nanometres can be employed and even combinations within one optical or optical/Doppler probe. Typical electrical power rating is 1 Watt per LED but employment of higher power devices are envisaged. Any LED with wavelength between about 550 nm and about 700 nm can in principle be employed.

The components and electronic circuitry included in the units described here are shown by way of example only in FIGS. 14-16. The circuitry shown in FIG. 14 controls the LEDs in the device shown in FIG. 9. The circuitry seen in FIG. 15 describes the Doppler ultrasound transmitter and FIG. 16 that of the receiver. The power amplifier and loudspeaker are not shown here. These circuits and the particular dimensions described in this document are to be treated as examples useful for implementing the invention. The transducer elements may be semi circular as described here or can be two rectangular slabs of suitable dimensions. The two piezoelectric elements maybe subdivided into, for example, 4 segments, opposite quadrants being connected together. This latter can reduce the effect of vessel orientation with respect to the breast checker geometry on Doppler signal detection.

The thickness of the piezoelectric elements determines their operating frequency, normally being half wavelength.

In the preferred embodiment of the invention described there is a microprocessor which allows reset of the light levels to the previous value to facilitate serial breast examinations, controls current through the LEDs, warns of low battery level and in the case of the clinical version of the breast checker interprets the Doppler signals in terms of advice to go for medical assessment. This can be a flashing light or a message on an LCD display. The microprocessor in the breast checker allows the light intensity from the probe to be stepped up automatically to the reset level in order to realize optimum contrast for each user. The reset level refers to intensity employed at last use the device. The device shuts down after a predetermined time.

In the preferred embodiment a capacitive [or mechanical] microswitch maintains low light level until the breast checker is in contact with the skin. This feature protects the eyes of users which might otherwise develop temporarily reduced visual sensitivity after viewing bright LEDs.

In the preferred embodiment a sensor to detect ambient light level and adjust LED brightness accordingly can be included.

It is likely that patients with locally advanced breast cancer might be monitored to demonstrate the efficacy of chemotherapy or angiogenesis inhibiting drugs on their tumor using the breast checker described in this application.

### REFERENCES

Bundred N., Levack P., Watmough D. J. , and Watmough J. A. [1986]. Preliminary results using computerized tele-Diaphanography for the investigation of breast disease. British J. Hospital Med. 37, 70-71.

Brittenden J, Watmough D J, Heys S D and Eremin O. 1995 Preliminary clinical evaluation of a combined optical Doppler ultrasound instrument for the detection of breast cancer. Brit. J Radiology 68, 1344-1348.

Watmough D.J. [1982]. Diaphanography; Mechanism responsible for the images. Acta. Radiologica Oncol. 21, 11-15.

1. A handheld battery powered apparatus for examining a body of living tissues, which tissues may contain a neoplasm, utilizing a light source for illuminating the tissues comprising a D shaped array of high intensity light emitting diodes overlaid by an optically transparent window, means for reproducing the intensity of the light, means for applying ultrasound to the neoplasm positioned in the centre of the array, means for receiving reflected ultrasound from blood vessels in the tissues also situated in the D shaped array, and a loudspeaker or other means for reproducing the blood flow signals.

2. An apparatus according to claim 1 wherein the means for applying ultrasound and means for receiving reflected ultrasonic signals comprises an ultrasonic Doppler blood flow detector whose piezoelectric transmitting and receiving elements are semicircular discs or rectangles positioned in the space provided between the light emitting diodes.

3. An apparatus according to claim 1 where the piezoelectric elements are attached to the optically and ultrasonically transparent window overlying the light emitting diodes.

4. An apparatus according to claim 1 comprising a switching device to which the said means for applying light and ultrasound to the tissues being interrogated can be selected.

5. An apparatus according to claim 1 where the time varying Doppler frequency shift which is electronically produced is revealed to the user via an audio system such as internal or external loudspeaker or headphones.

6. An apparatus according to claim 1 where the combined optical ultrasound Doppler device includes an array of 2 or more piezoelectric elements for blood flow detection and analysis, operating at ultrasonic frequencies between 2 and 20 MHz and where the light emitting diodes operate at a wavelength lying between 550 and 700 nm.

7. An apparatus according to claim 1 where the intensity of the optical radiation is electronically incremented in steps which are indicated digitally or otherwise to the user so that serial examinations lead to images of tissues comparison of which permits the user to spot changes to the appearance of the illuminated tissue surface caused by underlying pathology at an early stage in the disease process.

8. An apparatus according to claim 1 where the light level is held at a low level until a pressure or capacitive sensor incorporated in the device touches the skin whereupon the light level increases to operate at the preset level thus protecting the eyes of the user and allowing them to maintain visual sensitivity. The device is protected from overheating by a heat sink situated behind the light emitting diodes and an embedded thermostatic element which at a selected temperature interrupts the current driving the diodes.

9. An apparatus according to claim 1 where the optical features are included but the ultrasonic features are disabled or omitted.

10. An apparatus according to claim 1 where the device has an output socket connectable to an external recording system able to present an ultrasonic Doppler frequency shift spectra.

11. An apparatus as described in claim 1 but adapted to the examination of testicular tissues.

12. Apparatus according to claim 1 but where contrast media are injected into the vascular system to increase the sensitivity of the Doppler ultrasound and optical interrogation of tissues.

13. Apparatus according to claim 1 where transparent gel or lubricant for enhancing optical penetration of tissues is applied between the window and the skin and through which light and ultrasound are transmitted.

14. An apparatus for examining a body of living tissues, the apparatus comprising a light source for illuminating the tissues, the light source having a light emitting face substantially in the shape of an angular segment of a circle.

15. An apparatus according to claim 14, wherein the light emitting face is substantially semi-circular.

16. An apparatus according to claim 15, wherein the light emitting face is substantially D shaped.

17. An apparatus according to claim 14 wherein the light source comprises an array of light emitting diodes.

18. An apparatus according to claim 17, wherein the array of light emitting diodes is substantially D shaped.

19. An apparatus according to claim 14 wherein the light emitting face comprises a window which is transparent to radiation emitted from the light source.

20. An apparatus according to claim 14 further comprising a control for reproducing the intensity of the light from the light source.

21. An apparatus according to claim 14 further comprising a means for applying ultrasound to a neoplasm to be examined by the apparatus, a means for receiving reflected ultrasound from blood vessels in the tissues, and a means for generating an output to be analyzed by a user which reproduces blood flow signals derived from the reflected ultrasound signals.

22. An apparatus according to claim 21, wherein the means for applying ultrasound and the means for receiving reflected ultrasound are positioned within the area of the light emitting face.

23. An apparatus according to claim 22, wherein the means for applying ultrasound and the means for receiving reflected ultrasound are positioned within a centre of the light emitting face.

24. An apparatus according to claim 21 wherein the light source comprises an array of light emitting diodes and the means for applying ultrasound and means for receiving reflected ultrasound comprises an ultrasonic Doppler blood flow detector having piezoelectric transmitting and receiving elements positioned in a space provided between the light emitting diodes.

25. An apparatus according to claim 24 wherein the light emitting face comprises a window which is transparent to radiation emitted from the light source and the piezoelectric elements are attached to the window overlying the light emitting diodes.

26. An apparatus according to claim 21 further comprising a switching device to which the said means for applying light and ultrasound to the tissues being interrogated can be selected.

27. An apparatus according to claim 21 further comprising an audio system by means of which a time varying Doppler frequency shift which is electronically produced is revealed to the user.

28. An apparatus according to claim 21 where the light source comprises an array of light emitting diodes and the means for applying ultrasound and the means for receiving reflected ultrasound includes an array of two or more piezoelectric elements for blood flow detection and analysis, operating at ultrasonic frequencies between 2 and 20 MHz and where the light emitting diodes operate at a wavelength lying between 550 and 700 nm.

29. An apparatus according to claim 14 further comprising a controller for the light source whereby the intensity of the optical radiation is electronically incremented in steps which are indicated to the user.

30. An apparatus according to claim 14 further comprising a light level controller whereby the light level is held at a low level until a pressure or capacitive sensor incorporated in the device touches the skin whereupon the light level increases to operate at the preset level.

31. An apparatus according to claim 14 wherein the light source comprises an array of light emitting diodes and further comprising a heat sink situated behind the light emitting diodes and an embedded thermostatic element which at a selected temperature interrupts the current driving the diodes to protect the apparatus from overheating.

32. An apparatus according to claim 14 further comprising an output socket connectable to an external recording system able to present an ultrasonic Doppler frequency shift spectra.

33. An apparatus according to claim 14 further comprising a housing for the light source, the light emitting face having a linear edge adjacent to a linear side of the housing and a curved edge projecting away from the linear edge and located inwardly of a side of the housing, whereby the linear edge can be placed substantially against the skin of a user beneath the breast and substantially against the chest wall for illuminating the breast.

34. An apparatus according to claim 33 wherein the linear edge is about 5 mm inwardly of the linear side of the housing.

35. An apparatus according to claim 33 wherein the housing has an integral handle portion remote from the light emitting face.

36. Use of the apparatus according to claim 1 for the examination of breast or testicular tissues.

37. Use according to claim 36 wherein contrast media are injected into the vascular system to increase the sensitivity of the Doppler ultrasound and optical interrogation of tissues.

38. Use according to claim 36 wherein transparent gel or lubricant for enhancing optical penetration of tissues is applied between the window and the skin and through which light and ultrasound are transmitted.
